(19) United States
(12) Patent Application Publication
(10) Pub. No.: US 2006/0188897 A1
(43) Pub. Date: Aug. 24, 2006

(54) Transposon mediated differential hybridisation

(76) Inventors: Ian G. Charles, London (GB); Duncan J. Maskell, Cambridge (GB)

Correspondence Address: NIXON & VANDERHYE, PC, 901 NORTH GLEBE ROAD, 11TH FLOOR, ARLINGTON, VA, 22203, US

(21) Appl. No.: 11/220,910

(22) Filed: Sep. 8, 2005

Related U.S. Application Data
(63) Continuation of application No. 10/356,733, filed on Feb. 3, 2003, now abandoned.
Application No. 10/356,733 is a continuation-in-part of application No. 10/031,786, filed on Mar. 21, 2002, now abandoned.
(60) Provisional application No. 60/352,858, filed on Feb. 1, 2002.

(30) Foreign Application Priority Data
Jul. 26, 1999 (GB) 9917531.7

(51) Int. Cl. C12Q 1/68; C40B 40/08

(52) U.S. Cl. 435/6

(57) ABSTRACT

A method for identifying an essential gene of an organism comprises: (i) providing a Library of transposon mutants of the said organism; (ii) isolating polynucleotide sequences from the library which flank inserted transposons; (iii) hybridising the said polynucleotide sequences with a polynucleotide library from the said organism; and (iv) identifying a polynucleotide in the said polynucleotide library to which the said polynucleotide sequences do not hybridise, thereby to determine an essential gene of the organism.

### FIELD OF THE INVENTION

The invention relates to methods for the isolation of genes which are essential for the survival of an organism and to antibacterials, fungicides, antiparasitics, pesticides and herbicides.

### BACKGROUND TO THE INVENTION

Various strategies to generate and characterize mutations in a number or organisms have been described that rely on transposon mutagenesis. Such approaches depend on survival of the particular organism following mutagenesis and thus only detect mutants in which transposons have inserted into non-essential genes. Mutagenesis protocols have been developed for some conditional states, comparing in vitro growth with in vivo survival, and the Signature Tagged Mutagenesis (STM) approach has been particularly successful in identifying mutants important in pathogenicity. However, these conditional methods cannot detect mutants in genes that are essential for bacterial survival and which when mutated result in a lethal phenotype.

However, essential genes and in particular the proteins which they encode may be good substrates for use in screens for antibacterials, antiparasitics, fungicides, pesticides and herbicides. The increase in prevalence of antibiotic-resistant bacteria, for example, has renewed interest in the search for new targets for antibacterial agents. Essential genes and their protein products potentially represent such targets.

Additionally, there is an interest in the identification of conditional essential genes, that is genes which are essential for the survival of an organism in a particular environment. In the case of pathogenic bacteria, for example, these are genes which may be required for survival in the host. Such genes and the proteins which they encode may be good targets for use in screens for antibacterials. Bacteria which carry mutations in such genes may be useful in attenuated live vaccines.

### SUMMARY OF THE INVENTION

We have devised a general method to identify all the essential genes in a bacterial genome, using a transposon mutagenesis technique. We have called the technique Transposon Mediated Differential Hybridisation (TMDH). Essential genes are those genes which, when missing (eg. because of a chromosomal deletion) or mutated to render them non-functional, result in a lethal phenotype. That is, genes without which a bacterium cannot survive.

The technique can also be used for the identification of conditional essential genes. Conditional essential genes are those genes which are not absolutely essential for bacterial survival, but which are essential for survival under various conditional restraints. Examples of particular conditional restraints include survival at elevated temperatures and survival of a pathogen within its host.

According to the present invention there is thus provided a method for identifying an essential gene of an organism, comprising:
(i) providing a library of transposon mutants of the said organism;
(ii) isolating polynucleotide sequences from the library which flank inserted transposons;
(iii) hybridising the said polynucleotide sequences with a polynucleotide library from the said organism; and
(iv) identifying a polynucleotide in the said polynucleotide library to which the said polynucleotide sequences do not hybridise, thereby to determine an essential gene of the organism.
   The invention also provides:
a method for identifying a conditional essential gene of an organism comprising:
   (i) providing a first sample of a library of transposon mutants of the said organism (input library);
   (ii) providing a second sample of the library and subjecting that sample to a conditional restraint;
   (iii) collecting the mutants that survive the conditional restraint in step (ii) to give a new library (output library);
   (iv) carrying out a method for identifying an essential gene of an organism on the input library from step (i) and on the output library from step (iii), thereby to determine a conditional essential gene of the organism;
use of an essential or conditional essential gene identified by a method of the invention or a polypeptide encoded by a said gene, in a method for identifying an inhibitor of transcription and/or translation of that gene and/or activity of a polypeptide encoded by that gene;
a method for identifying:
   (i) an inhibitor of transcription and/or translation of an essential or conditional essential gene identified by a method of the invention; and/or
   (ii) an inhibitor of activity of a polypeptide encoded by a said gene, which method comprises determining whether a test substance can inhibit transcription and/or translation of a said gene and/or activity of a polypeptide encoded by a said gene;
an inhibitor identified by a method for identifying an inhibitor of transcription and/or translation of an essential or conditional essential gene identified by a method of the invention and/or activity of a polypeptide encoded by that gene;
an inhibitor of transcription and/or translation of an essential or conditional essential gene and/or activity of a polypeptide encoded by that gene;
an inhibitor of the invention, wherein the essential or conditional essential gene is a bacterial, fungal or eukaryotic parasite essential or conditional essential gene;
an inhibitor of the invention for use in a method of treatment of the human or animal body by therapy;
use of an inhibitor of the invention for the manufacture of a medicament for use in the treatment of a bacterial, fungal or eukaryotic parasite infection.
a pharmaceutical composition comprising an inhibitor of the invention and a pharmaceutically acceptable carrier or diluent;
a method of treating a host suffering from a bacterial, fungal or eukaryotic parasite infection, which comprises administering to the host a therapeutically effective amount of an inhibitor of the invention;
an inhibitor of the invention, wherein the essential or conditional essential gene is a bacterial, fungal or pest essential or conditional essential gene;
use of an inhibitor of the invention as a plant bacteriocide, fungicide or pesticide;
an inhibitor of the invention, wherein the essential or conditional essential gene is a plant conditional or essential gene;
use of an inhibitor according of the invention as a herbicide;
a method for identifying a conditional essential gene of an organism, wherein the organism is a bacterium and the conditional restraint is growth of that bacterium in its host;
a bacterium attenuated by a non-reverting mutation in one or more genes identified by a method for identifying a conditional essential gene of an organism;
a vaccine comprising a bacterium of the invention and a pharmaceutically acceptable carrier or diluent;
a bacterium of the invention for use in a method of vaccinating a human or animal;
use of a bacterium of the invention for the manufacture of a medicament for vaccinating a human or animal; and
a method of raising an immune response in a mammalian host, which comprises administering to the host a bacterium of the invention.

### BRIEF DESCRIPTION OF THE FIGURES

FIG. 1 shows a diagrammatic representation of one potential scheme for carrying out Transposon Mediated Differential Hybridisation (TMDH).

Genomic DNA isolated from a library of bacteria previously subjected to mutagenesis with a transposon is digested with left- and right-arm transposon-specific (TS) and gene-specific (GS) restriction endonucleases. For the transposon TnphoA, the left-arm restriction endonuclease pair may be DraI/HaeIII and the right arm pair may be HpaI/HaeIII.

Restriction fragments in the 200 to 600 base pair (bp) range are purified following gel eletrophoresis and vectorette units with compatible ends are ligated to the purified fragments. The resulting separate fragment panels (ie. the left-arm and right-arm panels) may be further purified at this stage.

Polymerase chain reaction (PCR) is carried out on the left-arm and right-arm fragment panels using primer pairs comprising an oligonucleotide specific for a transposon sequence and a second oligonucleotide specific for a vectorette sequence. The two panels of PCR fragments thus generated constitute the left- and right-arm consensus probes, representing sequences from the genes that have been disrupted by transposon insertion. The panels of PCR fragments can be radioactively labelled and used in hybridization experiments.

FIG. 2 shows that the left- and right-arm consensus probes can generate different signals. TMDH uses probes derived from left- and right-arm regions flanking the sites of transposon insertions. FIG. 2 outlines a theoretical situation where an essential gene (gene b) is flanked by two non-essential genes. In diagrams A and B, transposons have inserted into regions of the non-essential gene a. Both left- and right-arm consensus probes comprise mainly sequences from the non-essential gene a. However, in C, where the transposon has inserted towards the end of a, part of the resulting consensus right-arm probe may hybridise with the essential gene b. A similar situation can also occur for transposon insertion within the non-essential gene c, where a component of the left-arm consensus probe may hybridize with the essential gene b. The differential analysis of the hybridisation signals produced from the two probes allows an interpretation to be made in terms of whether or not the gene is essential.

FIG. 3 shows agarose gel electrophoresis of λ TnphoA transposon library left- and right-arm PCR products. Lanes 1 and 6, 100 bp ladder; lane 2, left-arm PCR products; lane 4, right-arm PCR products. Lanes 3 and 5, PCR of DNA from the host strain (E. coli XAC) with left-arm and right-arm PCR primers, respectively. Note the absence of any PCR product from the control lanes 3 and 5.

FIG. 4 shows hybridisation of consensus probes to a gridded array of E. coli open reading frames. In (a) hybridisation is shown of the ³³P-labelled left-arm probe to the Panorama Gene Array (Sigma-Genosys Ltd). The three fields contain 4290 PCR-amplified open reading frames representing all E. coli protein coding genes. A positive hybridisation signal corresponds to a gene that has been disrupted by transposon insertion, thereby identifying a non-essential gene.

In (b) hybridisation is shown of the ³³P-labelled right-arm probe with the Panorama Gene Arrays (Sigma-Genosys Ltd). The three fields contain 4290 PCR-amplified open reading frames representing all E. coli protein coding genes. A positive hybridisation signal corresponds to a gene that has been disrupted by transposon insertion, thereby identifying a non-essential gene.

### DESCRIPTION OF THE SEQUENCE LISTING

SEQ ID NO: 1 sets out the sequence of the T7 RNA polymerase site.

SEQ ID NO: 2 sets out the sequence of a primer for use in amplifying the T7 RNA polymerase site from the pT7Blue vector.

SEQ ID NO: 3 sets out the sequence of a primer for use in amplifying the T7 RNA polymerase site from the pT7Blue vector.

SEQ ID NO: 4 sets out the sequence of the PHO2 primer.

SEQ ID NO: 5 sets out the sequence of the INV1 primer.

### DETAILED DESCRIPTION OF THE INVENTION

The invention provides a method for identifying essential genes of an organism. Typically, the method requires the construction of a library of transposon mutants of a particular organism.

The library of transposon mutants can be used to generate a “consensus probe” which comprises a complex pool of polynucleotide sequences from the mutants in the library. The consensus probe comprises polynucleotide sequences which flank the transposon insertion sites and thus comprises sequences from genes that are non-essential. The particular method used to generate the consensus probe may allow the isolation of sequences from one or both regions flanking the transposons. Typically, two steps are used to generate consensus probes. Firstly, the sequences flanking the transposons are isolated and secondly, they are amplified.

The consensus probe is hybridized to polynucleotides from the organism used to generate the transposon-tagged mutants. Polynucleotides that do not hybridize to the consensus probe may correspond to genes that are essential for the survival of the organism in question.

Construction of a Library of Transposon Mutants

Typically, a library of transposon mutants is generated. Transposons, sometimes called transposable elements, are mobile polynucleotides. The term transposon is well known to those skilled in the art and includes classes of transposons that can be distinguished on the basis of sequence organisation, for example short inverted repeats at each end; directly repeated long terminal repeats (LTRs) at the ends; and polyA at 3′ ends of RNA transcripts with 5′ ends often truncated. Some types of virus also integrate into the host genome, for example retroviruses, and may therefore be used to generate libraries of insertion mutants. However, transposons are typically preferred to viruses because issues of safety related to pathogenicity may be avoided.

Any suitable transposon may be used for the generation of transposon libraries.

Suitable bacterial transposons include Tn3, γδ, Tn10, Tn5, TnphoA, Tn903, Tn917, Bacteriophage Mu and related viruses. Any of the above mentioned transposons may be used in a method of the invention. Preferred transposons are those which carry antibiotic resistance genes (which may be useful in identifying mutants which carry a transposon) including Tn5, Tn10 and TnphoA. For example, Tn10 carries a tetracycline resistance gene between its IS elements and Tn5 carries genes encoding polypeptides conferring resistance to kanamycin, streptomycin and bleomycin. It is of course possible to generate new transposons by inserting different combinations of antibiotic resistance genes between its IS elements or by altering the polynucleotide sequence of the transposon, for example by making a redundant base substitution in the coding region of an antibiotic resistance gene. It will be apparent that such transposons are included within the scope of the invention.

Suitable fungal transposable elements include the Ty1 element of Saccharomyces cerevisiae, the filamentous fungi elements (the filamentous fungi include agriculturally important plant pathogens such as Erysiphe graminis, Magnaporthe grisea) such as Fot1/Pogo-like and Tc1/Mariner-like elements (see Kempen and Kuck, 1998, Bioessays 20, 652-659 for a review of such elements).

Suitable plant elements include Ac/Ds, Tam3 and other Tam elements, cin4 and spm.

Suitable animal elements include P and hobo which may be used in Drosophila and Tc1 which can be used in Caenorhabditis elegans.

Libraries of transposon mutants may be generated according to any method known to those skilled in the art. For example, libraries of bacterial transposon mutants can be constructed using either plasmid or bacteriophage vectors containing the transposon and a selectable marker. Bacteriophage λ eg. λTnphoA can be used to infect a suitable recipient bacterial strain, for example E. coli XAC. This E. coli strain has a suppressor mutation which prevents the bacteriophage from replicating and subsequently lysing and also contains an antibiotic resistance gene to allow selection of colonies containing transposed chromosomal DNA. The vector contains mutation(s) preventing integration of the λ chromosome into the host (bacterial) chromosome and thus the growth of false positive colonies without a mutated E. coli gene is prevented. Cultures of the recipient strain are grown in enriched medium (eg. Luria Broth) and cells in mid log phase of growth are infected with the λ transposon vector for 1 hour at 37° C. Aliquots of the infected cells are plated out on L-agar supplemented with the appropriate selective antibiotic and grown overnight at 37° C. These colonies consitute a transposon library and can be further analysed by the TMDH procedure described in this application.

Growth of such libraries results in the generation of thousands of mutants and these result from mutations that are all, of necessity, in genes that when mutated do not result in the death of the cell ie. the non-essential genes. Typically, such a library will comprise at least one transposon insertion in at least 80%, preferably at least 90%, more preferably, at least 95% and most preferably at least 99% of non-essential genes.

Some regions of a particular genome may be inaccessible to insertion by a particular transposon, for example because of a particular secondary or tertiary structure which is inaccessible to a particular transposon. Thus it may be advantageous to combine two transposon libraries, thereby increasing the probability of obtaining transposon insertions in a greater number of genes. For example, in the case of bacterial libraries, Tn5 and Tn10 libraries for example, could be combined.

Generation of Consensus Probes

A consensus probe is generated from polynucleotide sequences that flank the transposons. The consensus probe may comprise polynucleotide sequences from one or both sides of any transposon. This will generally depend on the type of method used to generate the consensus probe. For example, inverse PCR may lead to the isolation of polynucleotide sequences from both sides of a transposon, whereas vectorette PCR typically leads to the isolation of polynucleotide sequence from one side of a transposon.

Generally flanking sequence will be isolated from at least 80%, preferably at least 90%, more preferably at least 95% and most preferably at least 99% of the mutants in a particular library, panel or pool, of mutants.

Any method known to those in the art may be used to isolate polynucleotide sequences flanking transposons and thus to generate consensus probes.

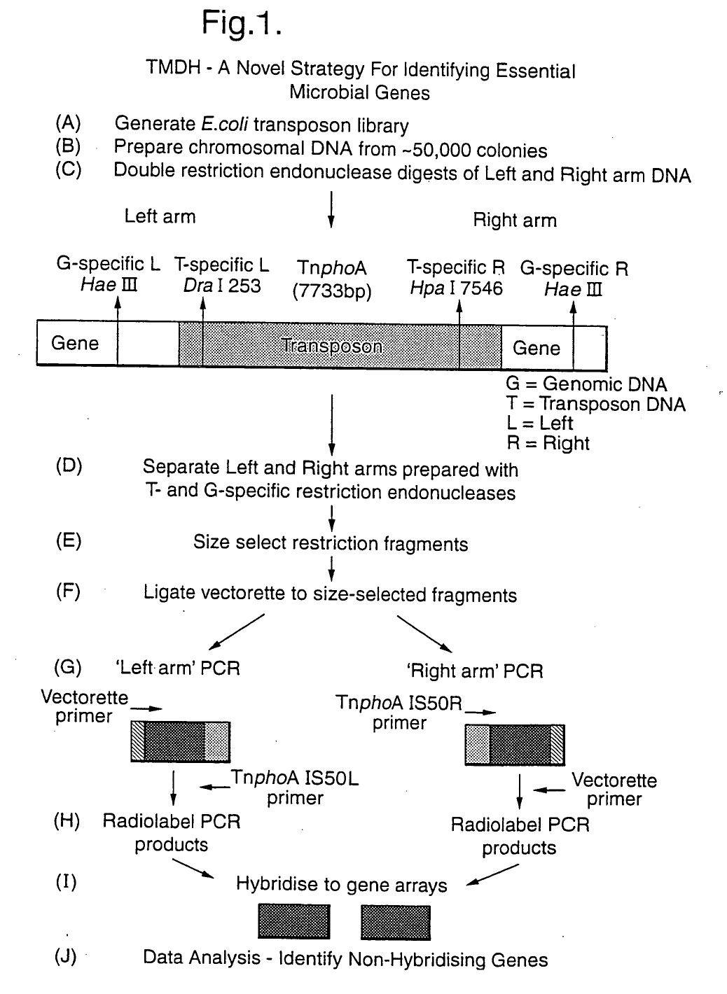

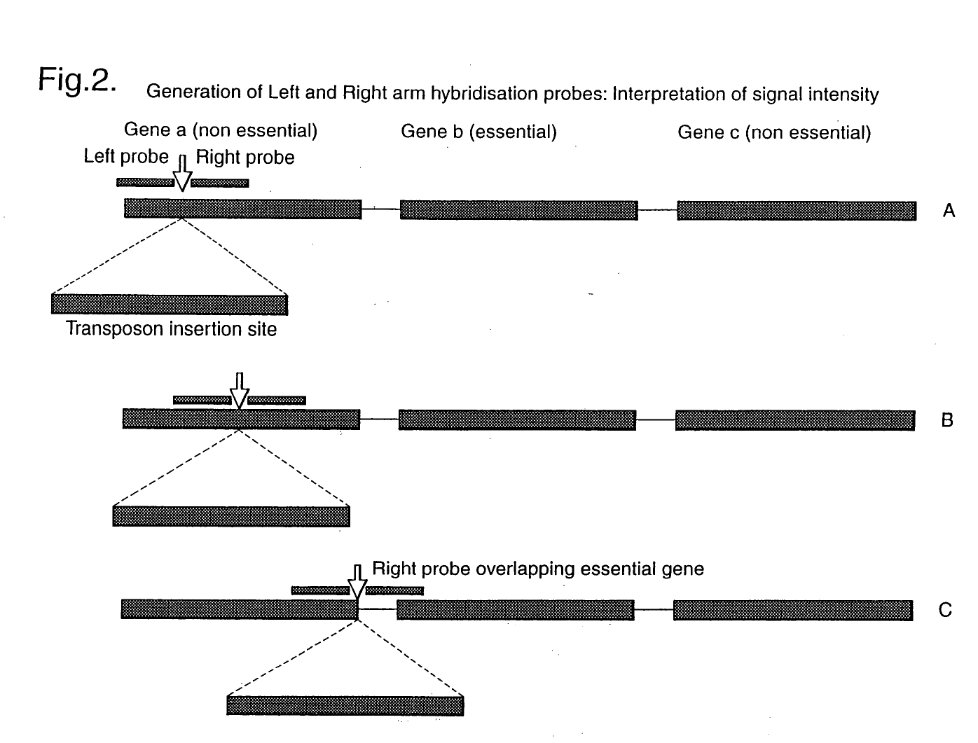

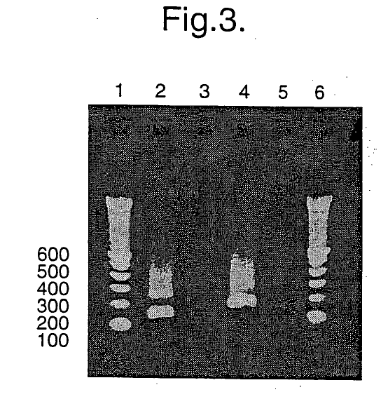

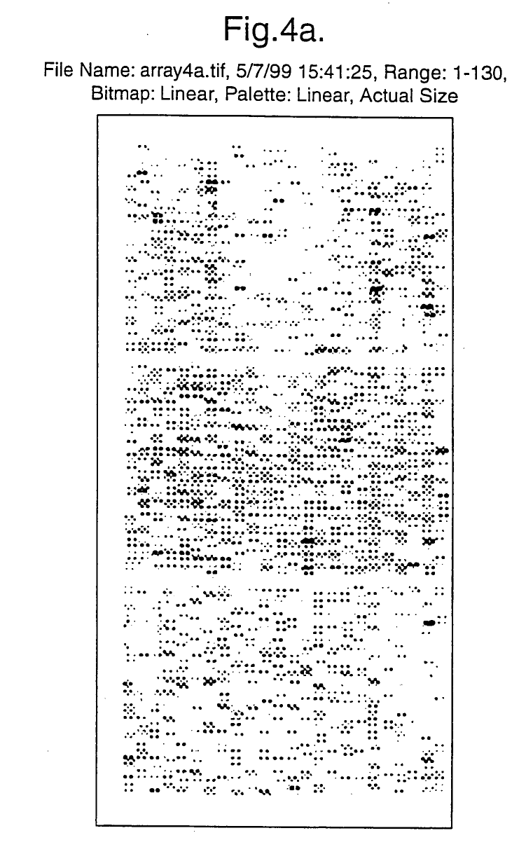

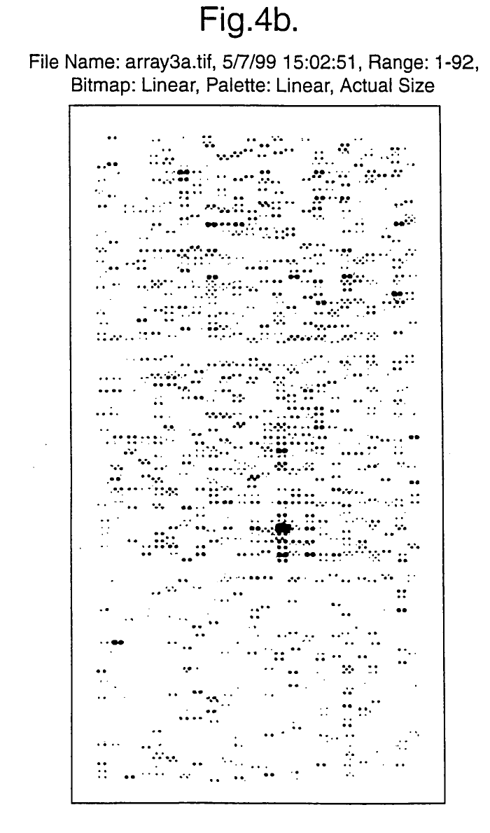

A preferred method involves the isolation of two consensus probes: a left-arm consensus probe (comprising sequences flanking the left hand sides of the transposons) and a right-arm consensus probe (comprising sequences flanking the right hand sides of the transposons). Each consensus probe is generally isolated by restriction endonuclease digestion, typically followed by an amplification step, for example PCR. Restriction endonuclease digestion may be followed by ligation of a linker such as a vectorette unit before the amplification step (FIG. 1).

In a preferred method of the invention, genomic DNA is isolated from a library of transposon mutants and digested with a first restriction endonuclease that cuts near the end of the transposon. Typically, suitable endonucleases have hexanucleotide recognition sequences. The exact restriction endonuclease used will depend on the sequence of the transposon which was used to generate the transposon-tagged library. These enzymes are referred to as the Transposon-specific (T-specific) endonucleases. In the case of TnphoA, suitable T-specific endonucleases are DraI, which cuts close to the left hand end of the transposon, and HpaI, which cuts close to the right hand end of the transposon (FIG. 1). Generally, an aliquot of the library is digested with the left hand T-specific endonuclease and a further aliquot is separately digested with the right hand T-specific endonuclease.

The resulting fragment pools may then be separately digested with a further restriction endonuclease, which will typically be different from the T-specific endonuclease. The second endonuclease, the Gene-specific (G-specific) endonuclease, is intended to cut somewhere in the genomic sequence that has been disrupted by the transposon. Generally, the G-specific endonuclease will have a four base pair recognition sequence and suitable examples are given in Table 1 below:

TABLE 1

Examples of 4 bp recognition type II
restriction endonucleases suitable for
use in TMDH

| | | | |
|---|---|---|---|
| Enzyme | Recognition Site | Enzyme | Recognition Site |

| | | | |
|---|---|---|---|
| AciI | C^{\|}CGC | MseI | T^{\|}TAA |
| | GGC_{\|}G | | AAT_{\|}T |
| AluI | AG^{\|}CT | MspI | C^{\|}CGG |
| | TC_{\|}GA | | GGC_{\|}C |
| BfaI | C^{\|}TAG | NlaIII | ^{\|}CATG |
| | GAT_{\|}C | | GTAC_{\|} |
| BstuI | CG^{\|}CG | RsaI | GT^{\|}AC |
| | GC_{\|}GC | | CA_{\|}TG |
| DpnI | ^{\|}GATC | Sau3a | ^{\|}GATC |
| | CTAG_{\|} | | CTAG_{\|} |
| HaeIII | GG^{\|}CC | TaqI | T^{\|}CGA |
| | CC_{\|}GG | | AGC_{\|}T |
| HinpI | G^{\|}CGC | Tsp509 | ^{\|}AATT |
| | CGC_{\|}G | | TTAA_{\|} |

In some cases it may be convenient to use the same restriction enzyme both as the T- and G-specific endonuclease, i.e. the same enzyme may be used to cut within the transposon and within the interrupted sequence. In addition, it may also be convenient to use the same enzyme to cut at both the left hand side and the right hand side of the transposon.

The resulting fragments may then be size selected. Typically fragments with a size of from approximately 200 to 600 bp are isolated, for example from a gel, and purified. The smaller the fragments isolated, the smaller the chance of the consensus probes including sequences from genes which lie next to genes which have been interrupted by transposons. Typically, the left- and right-arm pools of fragments are then amplified.

Amplification may be carried out by ligating linkers, preferably vectorette units, to the left- and right-arm fragment pools. If linkers are ligated to the left- and right-arm pools, the resulting fragments may be re-purified for example through a gel or by using spun-column chromatography. PCR may then carried out using the left- and right-arm pools of fragments as templates and a primer pair comprising an oligonucleotide specific for a transposon sequence and a second oligonucleotide specific for a linker (eg. a vectorette) sequence (FIG. 1g). The use of transposon- and vectorette-specific PCR primers results in the specific amplification of sequences that are adjacent to the sites of transposon insertion.

Alternatively, the left- and right-arm pools of fragments may be amplified by cycle primer extension. The use of a suitable labelled oligonucleotide primer can allow the amplification of sequences adjacent to the sites of transposon insertion. Those labelled amplified sequences can be used directly in hybridisation experiments.

Alternatively, the left- and right-arm pools may be amplified by inverse PCR (IPCR). Thus, the left- and right-arm pools of fragments may be self-ligated and subsequently amplified using transposon specific primers. When using IPCR techniques there is the possibility that, a “stuffer” fragment may ligate into the self-ligation reaction, which will be amplified along with the transposon-disrupted sequence. If this material were to be using in labelling experiments, the stuffer sequence could create non-specific background signal as it bound to the polynucleotide library. In order to remove this stuffer fragment, biotinylated primers can be used in the IPCR reaction. Following IPCR, the consensus sequences can be redigested with whichever enzyme was used to isolate the flanking sequences in the first place. This results in the release of the stuffer fragments and the consensus sequences may then be separated from the “stuffer” fragments using a magnetic-bead-streptavidin conjugate. The purified DNA can then be labeled and used to hybridize to polynucleotide libraries, for example a gridded array.

The techniques described above can therefore result in the isolation of sequences flanking both sides of the transposons. These pools of flanking fragments, the left- and right-arm consensus probes, may be used in hybridisation experiments to determine the essential genes.

Further methods for generating a consensus probe include the use of artificial transposons which comprise RNA polymerase binding site sequences. Such transposons may be used to generate transposon insertion libraries. The sequences flanking the transposons in such a library can be isolated by the addition of RNA polymerase to DNA from the transposon library which has been isolated, digested and size selected as described above. The RNA transcripts thus generated can be labelled and used in hybridisation experiments as described below. Alternatively, the RNA transcripts can be reverse transcribed and the complementary DNAs thus produced can be labelled and used in hybridisation experiments. The use of a transposon with different polymerase binding sites at each of its ends may allow for the isolation of left- and right-arm pools of fragments.

Additional methods for generating a consensus probe include, for example, splinkerette-PCR, targetted gene walking PCR, restriction site PCR, capture PCR, panhandle PCR and boomerang DNA amplification (for a review of these techniques see Hui et al., Cell Mol. Life Sci. 54 (1998) 1403-1411).

The techniques described above for the generation of a consensus probe typically require the digestion of genomic DNA isolated from the library of transposon mutants with a G-specific restriction endonuclease (for example, HaeIII in FIG. 1). It is possible that the particular G-specific endonuclease used in an experiment will not cut within the gene in which the transposon is inserted, or cuts at a large distance, for example more than 2 kb, away from the insertion site. Therefore sequences from these genes will not form part of the consensus probe. Thus the generation of consensus probes may be carried out several times, each time using different G-specific restriction endonucleases. The greater the number of enzymes used to make consensus probes, the greater the likelihood of sequences from non-essential genes being represented in the consensus probes. A similar result may be achieved by combining two or more of the techniques for generating consensus probes.

Hybridization of Consensus Probes to Polynucleotide Libraries

The sequences which comprise the consensus probes may be labelled for use as probes in hybridization experiments. Suitable labels include radioisotopes such as ³²P, ³³P or ³⁵S, enzyme labels or other labels such as biotin or digoxigenin or fluorescent labels. These labels may be detected using methods well known to those skilled in the art.

Generally the consensus probe is hybridized with polynucleotides isolated from the organism being studied. The polynucleotides used will typically be in the form of a library and generally be from a wild type organism. Genomic or cDNA libraries, for example, could be used. Polynucleotides in the library to which the consensus probes do not hybridize may comprise all or part of an essential gene.

Ideally, a library used in a hybridization experiment will be in the form of a gridded array. Gridded arrays typically comprise a different clone at every location on the array and preferably the array represents the whole of an organism's genome (if the array is a genomic DNA array) ie. it may represent the whole of a bacterial genome, for example. Alternatively, the array could be an expression array, in which case it would preferably comprise all messages from a particular organism. Particularly preferred libraries are those where each location of the gridded array represents a single open reading frame of the organism, wherein all the open reading frames from the organism are represented. In that way all protein coding polynucleotide sequences are represented. The advantage of using gridded arrays is that a whole genome may be analyzed in one experiment, very quickly and the clones to which the consensus probe does not hybridize are immediately available in a purified form. Additionally, in the case of an organism whose entire genome has been sequenced, for example E. coli or S. cerevisiae, the order of all open reading frames in the genome is known. Therefore, the order of all the open reading frames represented on a gridded array is known. This may be useful in interpreting hybridisation results, as is described below.

Hybridization experiments are typically carried out using two copies of the gridded array. In such experiments, the first array may be hybridized with a left-arm consensus probe, while the second array is hybridized with the corresponding right-arm consensus probe.

A location which on both the left- and right-arm arrays shows no hybridisation is likely to correspond to an essential gene. FIG. 2, however, shows that in some cases small regions of essential gene sequence may be isolated in a consensus probe in the event of a transposon inserting close to the end of a non-essential gene which lies adjacent to an essential gene. Thus essential genes may be capable of generating a small hybridisation signal on an array. An essential gene may give a hybridisation signal at a particular location only on one of the right and left arm arrays. Therefore not all clones on an array which give a positive signal should be classed as non-essential.

However, the amount of hybridisation seen for an essential gene will typically be much lower than that seen for an adjacent non-essential gene. This can be seen from FIG. 2 which shows two important aspects of TMDH. Firstly, it is desirable that as many different insertions are obtained for as many genes as possible in the genome under study. Secondly, the use of an array from an organism whose entire genome has been sequenced and therefore where the order of genes in the genome is known may be crucial in interpreting the results of hybridisations.

Identification of Conditional Essential Genes

The method may also be used for the identification of conditional essential genes. Conditional essential genes are those which are not absolutely essential for bacterial survival, but are essential for survival in particular environments eg. survival in a host (in the case of a pathogenic bacterium) or survival at elevated temperatures. Such environments are known as conditional restraints.

In order to isolate conditional essential genes, a library of transposon mutants is generated under control conditions (eg. growth at 37° C. in complete media). The library of mutants is then subjected to some conditional restraint. For example, the library of mutants can be inoculated in a suitable host, if it is a pathogen. Alternatively, the library of mutants can be grown at an elevated temperature. After the library of mutants has been subjected to the conditional restraint it can be recovered.

The library of mutants that have been exposed to the conditional restraint will lack mutants which carry transposons in those genes essential for growth under the conditional environment.

The control and conditional restraint libraries can be subjected to TMDH as described above. Optionally, right- and left-arm consensus probes from the control library are pooled and right- and left-arm consensus probes from the conditional restraint library are pooled. The two resulting pools may then be hybridised separately to polynucleotide libraries, preferably in the form of gridded arrays. Alternatively, if the pooling step is not carried out, four separate hybridisations will be necessary: control left-arm consensus probe; control right-arm consensus probe; conditional restraint left-arm consensus probe; and conditional restraint right-arm consensus probe.

Comparison of the results given with the control and the conditional restraint libraries will allow the identification of genes which permit survival in the conditional restraint. Genes identified as essential for survival in the conditional restraint library, but not identified as essential for survival under control conditions should represent genes that are essential for survival under the conditional restraint.

In the case of the analysis of conditional mutations in a pathogen, a library of Salmonella typhimurium transposon mutants, for example, can be used to infect a mouse. Following infection, bacteria target to livers and spleens and the course of infection can be conveniently followed by performing viable bacterial counts on those organs. The bacteria recovered from the livers and spleens can be grown on suitable plates. In the case of the conditional restraint at elevated temperature, a transposon-tagged library can be grown at 42° C.

Other conditional restraints include growth of antibiotic resistant bacteria in the present of antibiotics. This may reveal genes which are essential for antibiotic resistance. Such genes would be targets for drugs with the ability to lower bacterial resistance to particular antibiotics. Organisms could be grown in the presence of carcinogens, UV or other agents that cause oxidative stress and thus genes that confer resistance to growth under those conditions may be identified.

Verification of the Phenotype

Potential essential gene sequences and conditional essential gene sequences identified by the TMDH strategy may be verified using a method based on allelic exchange. This technique is particularly suitable for analysis of bacterial genes. PCR primers can be used to generate left- and right-arm sequences corresponding to the target gene sequence and ligated with a kanamycin-resistance encoding gene cassette. The resulting cassette can be introduced into a suicide vector, for example a plasmid-based vector, which is unable to replicate in a host bacterium.

In the case of a candidate essential gene, the resulting construct can be introduced into the bacterial strain from which the candidate gene originates. If the target gene is essential, it should be impossible to isolate allelic-exchange mutants that have a disrupted version of the target gene. In the case of a candidate conditional essential gene, the essential gene can be introduced into the bacterial strain from which the candidate gene originates. Allelic-exhange mutants can be isolated and subjected to growth under the conditional restraint. If the candidate gene is a conditional essential gene, it should not be possible for the allelic-exchange mutants to survive under the conditional restraint.

Similar experiments may be performed for other organisms

Bioinformatics

The use of bioinformatics may allow the rapid isolation of further essential and conditional essential genes. A gene identified in TMDH may be used to search databases containing sequence information from other species in order to identify orthologous genes from those species. Genes so identified can be tested for being essential or conditionally essential using the genetic techniques described above. For example, an E. coli gene is identified as essential using a method as described above. This may allow the identification of a putative orthologue from Salmonella. That Salmonella gene may be tested by allelic exchange and the construction of conditional mutants in Salmonella as described above. Further orthologues may be identified in more distantly related organisms, for example from Plasmodium species.

Suitable bioinformatics programs are well known to those skilled in the art. For example, the Basic Local Alignment Search Tool (BLAST) program (Altschul et al., 1990, J. Mol. Biol. 215, 403-410. and Altschul et al., 1997, Nucl. Acids Res. 25, 3389-3402.) may be used. Suitable databases for searching are for example, EMBL, GENBANK, TIGR, EBI, SWISS-PROT and trEMBL.

Organisms Useful in the Invention

Organisms that may be used in the invention are those for which it is possible to carry out transposon mutagenesis and thus, those that can give rise to a library of transposon mutants. Clearly, if the genome is bigger, more mutants will have to be produced in order to give a better chance of achieving saturation mutagenesis.

Suitable organisms include prokaryotic and eukaryotic organisms. Suitable prokaryotes include bacteria. Preferred bacteria are those which are animal or human or plant pathogens.

The bacteria used may be Gram-negative or Gram-positive. The bacteria may be for example, from the genera Escherichia, Salmonella, Vibrio, Haemophilus, Neisseria, Yersinia, Bordetella, Brucella, Shigella, Klebsiella, Enterobacter, Serracia, Proteus, Vibrio, Aeromonas, Pseudomonas, Acinetobacter, Moraxella, Flavobacterium, Actinobacillus, Staphylococcus, Streptococcus, Mycobacteriurn, Listeria, Clostridium, Pasteurella, Helicobacter, Campylobacter, Lawsonia, Mycoplasma, Bacillus, Agrobacterium, Rhizobium, Erwinia or Xanthomonas.

Examples of some of the above mentioned genera are Escherichia coli—a cause of diarrhoea in humans; Salmonella typhimurium—the cause of salmonellosis in several animal species; Salmonella typhi—the cause of human typhoid fever; Salmonella enteritidis—a cause of food poisoning in humans; Salmonella choleraesuis—a cause of salmonellosis in pigs; Salmonella dublin—a cause of both a systemic and diarrhoeal disease in cattle, especially of new-born calves; Haemophilus influenzae—a cause of meningitis; Neisseria gonorrhoeae—a cause of gonorrhoea; Yersinia enterocolitica—the cause of a spectrum of diseases in humans ranging from gastroenteritis to fatal septicemic disease; Bordetella pertussis—the cause of whooping cough; Brucella abortus—a cause of abortion and infertility in cattle and a condition known as undulant fever in humans; Vibrio cholerae—a cause of cholera; Clostridium tetani—a cause of tetanus; Bacillus anthracis—a cause of anthrax.

Suitable eukaryotes include fungi, plants and animals. Preferred eukaryotes include animal or human parasites and plant pests.

Suitable fungi include the animal pathogens including Candida albicans—a cause of thrush, Trichophyton spp.—a cause of ringworm in children, athlete's foot in adults. Other suitable fungi include the plant pathogens Phytophthora infestans, Plasmopara viticola, Peronospora spp., Saprolegnia spp., Erysiphe spp., Ceratocystis ulmi, Monilinia fructigena, Venturia inequalis, Claviceps purpurea, Diplocarpon rosae, Puccinia graminis, Ustilago avenae.

Suitable animal parasites include Plasmodium spp., Trypanasoma spp., Giarda spp., Trichomonas spp. and Schistosoma spp. Other animal parasites include the various platyhelminth, nematode and annelid parasites.

Suitable plant pests include insects, nematodes and molluscs such as slugs and snails.

Suitable plants include monoctyledons and dicotyledons.

Preferred organisms are those for which the entire genome has been sequenced and therefore for which it may be possible to construct gridded arrays covering the entire genome or all of the open reading frames.

Screens for Inhibitors of Essential and Conditional Essential Genes

Essential and conditional essential genes of bacteria and the polypeptides which they encode may represent targets for antibacterial substances. Similarly essential and conditional essential genes of fungi and eukaryotic parasites, pests and plants and the proteins which they encode may represent targets for fungicides, antiparasitics, pesticides and herbicides respectively. Fungicides may have both animal and plant applications.

Furthermore, if a particular gene is essential or conditionally essential for a number of different bacteria, fungi, parasites, pests or plants, that gene and the polypeptide it encodes may represent a target for substances with a broad-spectrum of activity.

An essential or conditional essential gene identified by a method as described above and the polypeptide which it encodes may be used in a method for identifying an inhibitor of transcription and/or translation of the gene and/or activity of the polypeptide encoded by the gene. Such a substance may be referred to as an inhibitor of an essential or conditional essential gene. Thus, an inhibitor of an essential or conditional essential gene is a substance which inhibits expression and/or translation of that essential gene and/or activity of the polypeptide encoded by that essential or conditional essential gene.

Any suitable assay may be carried out to determine whether a test substance is an inhibitor of an essential or conditional essential gene. For example, the promoter of an essential or conditional essential gene may be linked to a coding sequence for a reporter polypeptide. Such a construct may be contacted with a test substance under conditions in which, in the absence of the test substance expression of the reporter polypeptide would occur. This would allow the effect of the test substance on expression of the essential or conditional essential gene to be determined.

Substances which inhibit translation of an essential or conditional essential gene may be isolated, for example, by contacting the mRNA of the essential or conditional essential gene with a test substance under conditions that would permit translation of the mRNA in the absence of the test substance. This would allow the effect of the test substance on translation of the essential or conditional essential gene to be determined.

Substances which inhibit activity of a polypeptide encoded by the essential gene may be isolated, for example, by contacting the polypeptide with a substrate for the polypeptide and a test substance under conditions that would permit activity of the polypeptide in the absence of the test substance. This would allow the effect of the test substance on activity of the polypeptide encoded by the essential or conditional essential gene to be determined.

Suitable control experiments can be carried out. For example, a putative inhibitor should be tested for its activity against other promoters, mRNAs or polypeptides to discount the possibility that it is a general inhibitor of gene transcription, translation or polypeptide activity.

Test Substances

Suitable test substances for inhibitors of essential or conditional essential genes include combinatorial libraries, defined chemical entities, peptides and peptide mimetics, oligonucleotides and natural product libraries. The test substances may be used in an initial screen of, for example, ten substances per reaction, and the substances of batches which show inhibition tested individually. Furthermore, antibody products (for example, monoclonal and polyclonal antibodies, single chain antibodies, chimaeric antibodies and CDR-grafted antibodies) may be used.

Inhibitors of Essential Genes

An inhibitor of an essential or conditional essential gene is one which inhibits expression and/or translation of that essential gene and/or activity of the polypeptide encoded by that essential or conditional gene. Preferred substances are those which inhibit essential gene expression and/or translation and/or activity by at least 10%, at least 20%, at least 30%, at least 40%, at least 50%, at least 60%, at least 70%, at least 80%, at least 90%, at least 95% or at least 99% at a concentration of the inhibitor of 1 μgml⁻¹, 10 μgml⁻¹, 100 μgml⁻¹, 500 μgml⁻¹, 1 mgml⁻¹, 10 mgml⁻¹, 100 mg ml⁻¹. The percentage inhibition represents the percentage decrease in expression and/or translation and/or activity in a comparison of assays in the presence and absence of the test substance. Any combination of the above mentioned degrees of percentage inhibition and concentration of inhibitor may be used to define an inhibitor of the invention, with greater inhibition at lower concentrations being preferred.

Test substances which show activity in assays such as those described above can be tested in in vivo systems, such as an animal model of infection for antibacterial activity or a plant model for herbicidal activity. Thus, candidate inhibitors could be tested for their ability to attenuate bacterial infections in mice in the case of an antibacterial or for their ability to inhibit growth of plants in the case of a herbicide.

Therapeutic Use

Inhibitors of bacterial, fungal or eukaryotic parasite essential or conditional essential genes may be used in a method of treatment of the human or animal body by therapy. In particular such substances may be used in a method of treatment of a bacterial, fungal or eukaryotic parasite infection. Such substances may also be used for the manufacture of a medicament for use in the treatment of a bacterial, fungal or eukaryotic parasite infections The condition of a patient suffering from such an infection can be improved by administration of an inhibitor. A therapeutically effective amount of an inhibitor may be given to a human patient in need thereof.

Inhibitors of bacterial, fungal or eukaryotic parasite essential or conditional essential genes may be administered in a variety of dosage forms. Thus, they can be administered orally, for example as tablets, troches, lozenges, aqueous or oily suspensions, dispersible powders or granules. The inhibitors may also be administered parenterally, either subcutaneously, intravenously, intramuscularly, intrasternally, transdermally or by infusion techniques. The inhibitors may also be administered as suppositories. A physician will be able to determine the required route of administration for each particular patient.

The formulation of an inhibitor for use in preventing or treating a bacterial or fungal infection will depend upon factors such as the nature of the exact inhibitor, whether a pharmaceutical or veterinary use is intended, etc. An inhibitor may be formulated for simultaneous, separate or sequential use.

An inhibitor is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol;

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of an inhibitor is administered to a patient. The dose of an inhibitor may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific inhibitor, the age, weight and conditions of the subject to be treated, the type and severity of the degeneration and the frequency and route of administration. Preferably, daily dosage levels are from 5 mg to 2 g.

Live Attenuated Vaccines

The principle behind vaccination is to induce an immune response in the host thus providing protection against subsequent challenge with a pathogen. This may be achieved by inoculation with a live attenuated strain of the pathogen, i.e. a strain having reduced virulence such that it does not cause the disease caused by the virulent pathogen. Bacteria which carry mutations in conditional essential genes required for survival in a host isolated according to the methods described above may be good candidates for use in live attenuated vaccines.

The mutations introduced into the bacterial vaccine generally knock-out the function of the gene completely. This may be achieved either by abolishing synthesis of any polypeptide at all from the gene or by making a mutation that results in synthesis of non-functional polypeptide. In order to abolish synthesis of polypeptide, either the entire gene or its 5′-end may be deleted. A deletion or insertion within the coding sequence of a gene may be used to create a gene that synthesises only non-functional polypeptide (e.g. polypeptide that contains only the N-terminal sequence of the wild-type protein).

The bacterium may have mutations in one or more, for example two, three or four conditional essential genes. The mutations are non-reverting mutations. These are mutations that show essentially no reversion back to the wild-type when the bacterium is used as a vaccine. Such mutations include insertions and deletions. Insertions and deletions are preferably large, typically at least 10 nucleotides in length, for example from 10 to 600 nucleotides. Preferably, the whole coding sequence is deleted.

The bacterium used in the vaccine preferably contains only defined mutations, i.e. mutations which are characterised. It is clearly undesirable to use a bacterium which has uncharacterised mutations in its genome as a vaccine because there would be a risk that the uncharacterised mutations may confer properties on the bacterium that cause undesirable side-effects.

The attenuating mutations may be introduced by methods well known to those skilled in the art. Appropriate methods include cloning the DNA sequence of the wild-type gene into a vector, e.g. a plasmid, and inserting a selectable marker into the cloned DNA sequence or deleting a part of the DNA sequence, resulting in its inactivation. A deletion may be introduced by, for example, cutting the DNA sequence using restriction enzymes that cut at two points in or just outside the coding sequence and ligating together the two ends in the remaining sequence with an antibiotic resistance determinant. A plasmid carrying the inactivated DNA sequence can be transformed into the bacterium by known techniques such as electroporation or conjugation for example. It is then possible by suitable selection to identify a mutant wherein the inactivated DNA sequence has recombined into the chromosome of the bacterium and the wild-type DNA sequence has been rendered non-functional by homologous recombination.

The attenuated bacterium of the invention may be genetically engineered to express an antigen that is not expressed by the native bacterium (a “heterologous antigen”), so that the attenuated bacterium acts as a carrier of the heterologous antigen. The antigen may be from another organism, so that the vaccine provides protection against the other organism. A multivalent vaccine may be produced which not only provides immunity against the virulent parent of the attenuated bacterium but also provides immunity against the other organism. Furthermore, the attenuated bacterium may be engineered to express more than one heterologous antigen, in which case the heterologous antigens may be from the same or different organisms.

The heterologous antigen may be a complete protein or a part of a protein containing an epitope. The antigen may be from a virus, prokaryote or a eukaryote, for example another bacterium, a yeast, a fungus or a eukaryotic parasite. The antigen may be from an extracellular or intracellular protein. More especially, the antigenic sequence may be from E. coli, tetanus, hepatitis A, B or C virus, human rhinovirus such as type 2 or type 14, herpes simplex virus, poliovirus type 2 or 3, foot-and-mouth disease virus, influenza virus, coxsackie virus or Chlamydia trachomatis. Useful antigens include non-toxic components of E. coli heat labile toxin, E. coli K88 antigens, ETEC colonization factor antigens, P.69 protein from B. pertussis and tetanus toxin fragment C.

The DNA encoding the heterologous antigen is expressed from a promoter that is active in vivo. Two promoters that have been shown to work well in Salmonella are the nirB promoter and the htrA promoter. For expression of the ETEC colonization factor antigens, the wild-type promoters could be used.

A DNA construct comprising the promoter operably linked to DNA encoding the heterologous antigen may be made and transformed into the attenuated bacterium using conventional techniques. Transformants containing the DNA construct may be selected, for example by screening for a selectable marker on the construct. Bacteria containing the construct may be grown in vitro before being formulated for administration to the host for vaccination purposes.

The vaccine may be formulated using known techniques for formulating attenuated bacterial vaccines. The vaccine is advantageously presented for oral administration, for example in a lyophilised encapsulated form. Such capsules may be provided with an enteric coating comprising, for example, Eudragate “S” (Trade Mark), Eudragate “L” (Trade Mark), cellulose acetate, cellulose phthalate or hydroxypropylmethyl cellulose. These capsules may be used as such, or alternatively, the lyophilised material may be reconstituted prior to administration, e.g. as a suspension. Reconstitution is advantageously effected in a buffer at a suitable pH to ensure the viability of the bacteria. In order to protect the attenuated bacteria and the vaccine from gastric acidity, a sodium bicarbonate preparation is advantageously administered before each administration of the vaccine. Alternatively, the vaccine may be prepared for parenteral administration, intranasal administration or intramuscular administration.

The vaccine may be used in the vaccination of a mammalian host, particularly a human host but also an animal host. An infection caused by a microorganism, especially a pathogen, may therefore be prevented by administering an effective dose of a vaccine prepared according to the invention. The dosage employed will ultimately be at the discretion of the physician, but will be dependent on various factors including the size and weight of the host and the type of vaccine formulated. However, a dosage comprising the oral administration of from 10⁷ to 10¹¹ bacteria per dose may be convenient for a 70 kg adult human host.

Agricultural Use

Inhibitors of bacterial, fungal and pest essential or conditional essential genes may be administered to plants in order to prevent or treat bacterial, fungal or pest infections; the term pest includes any animal which attacks a plant. Thus inhibitors of the invention may be useful as pesticides. Inhibitors of plant essential or conditional essential genes may be administered to plants in order to reduce or stop plant growth, that is to act as a herbicide.

The inhibitors of the present invention are normally applied in the form of compositions together with one or more agriculturally acceptable carriers or diluents and can be applied to the crop area or plant to be treated, simultaneously or in succession with further compounds.

The inhibitors of the invention can be selective herbicides, bacteriocides, fungicides or pesticides or mixtures of several of these preparations, if desired together with further carriers, surfactants or application-promoting adjuvants customarily employed in the art of formulation. Suitable carriers and diluents correspond to substances ordinarily employed in formulation technology, e.g. natural or regenerated mineral substances, solvents, dispersants, wetting agents, tackifiers, binders or fertilizers.

A preferred method of applying active ingredients of the present invention or an agrochemical composition which contains at least one of the active ingredients is leaf application. The number of applications and the rate of application depend on the intensity of infestation by the pathogen. However, the active ingredients can also penetrate the plant through the roots via the soil (systemic action) by impregnating the locus of the plant with a liquid composition, or by applying the compounds in solid form to the soil, e.g. in granular form (soil application). The active ingredients may also be applied to seeds (coating) by impregnating the seeds either with a liquid formulation containing active ingredients, or coating them with a solid formulation. In special cases, further types of application are also possible, for example, selective treatment of the plant stems or buds.

The active ingredients are used in unmodified form or, preferably, together with the adjuvants conventionally employed in the art of formulation, and are therefore formulated in known manner to emulsifiable concentrates, coatable pastes, directly sprayable or dilutable solutions, dilute emulsions, wettable powders, soluble powders, dusts, granulates, and also encapsulations, for example, in polymer substances. Like the nature of the compositions, the methods of application, such as spraying, atomizing, dusting, scattering or pouring, are chosen in accordance with the intended objectives and the prevailing circumstances. Advantageous rates of application are normally from 50 g to 5 kg of active ingredient (a.i.) per hectare (“ha”, approximately 2.471 acres), preferably from 100 g to 2 kg a.i./ha, most preferably from 200 g to 500 g a.i./ha.

The formulations, compositions or preparations containing the active ingredients and, where appropriate, a solid or liquid adjuvant, are prepared in known manner, for example by homogeneously mixing and/or grinding active ingredients with extenders, for example solvents, solid carriers and, where appropriate, surface-active compounds (surfactants).

Suitable solvents include aromatic hydrocarbons, preferably the fractions having 8 to 12 carbon atoms, for example, xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol, monomethyl or monoethyl ether, ketones such as cyclohexanone, strongly polar solvents such as N-methyl-2-pyrrolidone, dimethyl sulfoxide or dimethyl formamide, as well as epoxidized vegetable oils such as epoxidized coconut oil or soybean oil; or water.

The solid carriers used e.g. for dusts and dispersible powders, are normally natural mineral fillers such as calcite, talcum, kaolin, montmorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite; and suitable nonsorbent carriers are materials such as calcite or sand. In addition, a great number of pregranulated materials of inorganic or organic nature can be used, e.g. especially dolomite or pulverized plant residues.

Depending on the nature of the active ingredient to be used in the formulation, suitable surface-active compounds are nonionic, cationic and/or anionic surfactants having good emulsifying, dispersing and wetting properties. The term “surfactants” will also be understood as comprising mixtures of surfactants.

Suitable anionic surfactants can be both water-soluble soaps and water-soluble synthetic surface-active compounds.

Suitable soaps are the alkali metal salts, alkaline earth metal salts or unsubstituted or substituted ammonium salts of higher fatty acids (chains of 10 to 22 carbon atoms), for example the sodium or potassium salts of oleic or stearic acid, or of natural fatty acid mixtures which can be obtained for example from coconut oil or tallow oil. The fatty acid methyltaurin salts may also be used.

More frequently, however, so-called synthetic surfactants are used, especially fatty sulfonates, fatty sulfates, sulfonated benzimidazole derivatives or alkylarylsulfonates.

The fatty sulfonates or sulfates are usually in the form of alkali metal salts, alkaline earth metal salts or unsubstituted or substituted ammoniums salts and have a 8 to 22 carbon alkyl radical which also includes the alkyl moiety of alkyl radicals, for example, the sodium or calcium salt of lignonsulfonic acid, of dodecylsulfate or of a mixture of fatty alcohol sulfates obtained from natural fatty acids. These compounds also comprise the salts of sulfuric acid esters and sulfonic acids of fatty alcohol/ethylene oxide adducts. The sulfonated benzimidazole derivatives preferably contain 2 sulfonic acid groups and one fatty acid radical containing 8 to 22 carbon atoms. Examples of alkylarylsulfonates are the sodium, calcium or triethanolamine salts of dodecylbenzenesulfonic acid, dibutylnaphthalenesulfonic acid, or of a naphthalenesulfonic acid/formaldehyde condensation product. Also suitable are corresponding phosphates, e.g. salts of the phosphoric acid ester of an adduct of p-nonylphenol with 4 to 14 moles of ethylene oxide.

Non-ionic surfactants are preferably polyglycol ether derivatives of aliphatic or cycloaliphatic alcohols, or saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols.

Further suitable non-ionic surfactants are the water-soluble adducts of polyethylene oxide with polypropylene glycol, ethylenediamine propylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit.

Representative examples of non-ionic surfactants are nonylphenolpolyethoxyethanols, castor oil polyglycol ethers, polypropylene/polyethylene oxide adducts, tributylphenoxypolyethoxyethanol, polyethylene glycol and octylphenoxyethoxyethanol. Fatty acid esters of polyoxyethylene sorbitan and polyoxyethylene sorbitan trioleate are also suitable non-ionic surfactants.

Cationic surfactants are preferably quaternary ammonium salts which have, as N-substituent, at least one C₈-C₂₂ alkyl radical and, as further substituents, lower unsubstituted or halogenated alkyl, benzyl or lower hydroxyalkyl radicals. The salts are preferably in the form of halides, methylsulfates or ethylsulfates, e.g. stearyltrimethylammonium chloride or benzyldi(2-chloroethyl)ethylammonium bromide.

The surfactants customarily employed in the art of formulation are described, for example, in “McCutcheon's Detergents and Emulsifiers Annual”, MC Publishing Corp. Ringwood, N.J., 1979, and Sisely and Wood, “Encyclopaedia of Surface Active Agents,” Chemical Publishing Co., Inc. New York, 1980.

The agrochemical compositions usually contain from about 0.1 to about 99% preferably about 0.1 to about 95%, and most preferably from about 3 to about 90% of the active ingredient, from about 1 to about 99.9%, preferably from about 1 to 99%, and most preferably from about 5 to about 95% of a solid or liquid adjuvant, and from about 0 to about 25%, preferably about 0.1 to about 25%, and most preferably from about 0.1 to about 20% of a surfactant.

Whereas commercial products are preferably formulated as concentrates, the end user will normally employ dilute formulations.

### EXAMPLES

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

### Example 1

A flow diagram outlining the TMDH procedure in shown in FIG. 1. Following the generation of a transposon library, DNA is purified from approximately 20 000 colonies (FIGS. 1a and 1b). In order to generate probes for the differential hybridization, gene sequences flanking the site of transposon insertion are recovered by a strategy involving double restriction endonuclease digestion (FIG. 1c). Left- and right-arm fragments in the 200 to 600 bp size range are purified by gel eletrophoresis (FIGS. 1d and 1e) and vectorette units ligated onto the ends (FIG. 1f).

In order to generate a specific probe population for subsequent hybridisation to the gene array filter, PCR is carried out with primer pairs specific for the transposon and the vectorette (FIG. 1g). FIG. 3 shows a gel analysis of the PCR amplification of left- and right-arms generated using this approach. The PCR step is designed to amplify only those sequences that have been disrupted by transposon insertion (FIG. 3, tracks 2 and 4). The effectiveness of this step is seen from analysis of tracks 3 and 5, where DNA from an E. coli isolate not harbouring a transposon is subjected to PCR with the same primers and results in no detectable amplification. Following amplification, the two probe populations produced from the left- and right-arms are radioactively labelled (FIG. 1h) and hybridized to an E. coli gridded array library (FIG. 1i).

FIGS. 4a and 4b shows the result produced following hybridisation with the left- and right-arm probes. A positive hybridisation signal on the array corresponds to a gene that has been disrupted by transposon insertion and is consequently unlikely to be essential.

### Example 2

The following experiments were carried out to give experimental details of three different approaches we have used to generate transposon-specific probes (consensus probes) for use in the TMDH technique.

(i) Cloning of a DNA-Dependent T7 RNA Polymerase Site into a Transposon Vector

DNA-dependent T7 RNA Polymerase sites have been incorporated into many plasmid vectors as a convenient means of generating RNA templates in a highly specific and regulated manner. These RNA products have been termed ‘run-off transcripts’. In order to use labeled run-off RNA transcripts in the TMDH protocol, we have engineered a DNA-dependent T7 RNA polymerase site into the transposon EZ::TN pMOD <MCS> vector (Epicentre Technologies). The RNA polymerase site has been engineered into the multiple cloning site (MCS). Following transposition, this novel transposon will allow the generation of specific fragments of RNA corresponding to the parts of gene(s) directly flanking the site of transposon insertion. Labeled probes generated in this fashion can be used to hybridise to polynucleotide libraries, for example gridded arrays, as described above (see the “Hybridization of consensus probes to polynucleotide libraries” section of the description).

The core DNA-dependent T7 RNA polymerase site from pT7Blue vector (Novagen):

5′-TAATACGACTCACTATAGGG-3′ (SEQ ID NO: 1)

was amplified together with 80 bp 5′-sequence in order to incorporate any flanking recognition motifs (bases 2830-62). The following primers were used:

(SEQ ID NO: 2)

1. 5′-CCGGCTCGTGTCGACTGTGGAATTG-3′ (2830-2854);

(SEQ ID NO: 3)

2. 5′-CTGCAGGCATGCAAGCTTTCCCTATAG-3′ (62-35),

Primer 1 (SEQ ID NO: 2) has a SalI site (underlined); and primer 2 (SEQ ID NO: 3) a HindIII site.

PCR was performed using pT7Blue vector as template and primer pairs 1&2 and 3&4 using the following parameters: 95° C. 5 min.; (94° C. 1 min.; 55° C. 1 min.; 72° C. 1 min.) for 30 cycles; 72° C. 5 min. final extension. PCR products were gel extracted (Qiagen) and cloned into the TOPO cloning vector (Invitrogen).

PCR product from primer pairs 1&2 was cut from TOPO with SalI and HindIII, cloned into EZ::TN pMOD <MCS> vector (Epicentre Technologies), transformed into JM109 cells (Promega) and selected on ampicillin.

Sequencing was performed to confirm the presence of the DNA-dependent T7 RNA polymerase site.

RNA was generated by in vitro transcription using the RiboMAX large scale RNA production system (Promega). 5 μg of DNA (EZ:TN vector with the cloned T7 promoter site) was digested with Afl III for 1 h at 37° C. and purified on a QIAquick column (Qiagen). Prior to RNA generation, the DNA sample was blunt-ended by treatment with 5 units of Klenow polymerase at 22° C. for 15 min.

RNA run-off transcripts were generated following the addition of nucleotide mix and T7 RNA polymerase to the reaction (30 μl of 100 mM mix of rNTPs and 10 μl T7 RNA polymerase). The reaction was incubated at 37° C. for 4 h. The AflIII digested DNA template produced an RNA transcript of 200 bp, demonstrating that the cloned T7 RNA polymerase site insert was functional.

For use in the TMDH protocol, a transposon library (generated with the EZ:TN transposon containing the cloned T7 promoter site) will be generated. DNA will be isolated, digested using the restriction endonucleases described, and size selected. Run off RNA transcripts generated from the cloned T7 promoter will be labeled and used to hybridize to polynucleotide libraries, typically in the form of gridded arrays.

(ii) Generating Transposon Specific Probes by Inverse PCR

We have devised an improved method to generate transposon specific probes by inverse PCR for use in TMDH protocols. The following example was carried out on DNA isolated from a TnphoA transposon mutagenesis experiment.

Genomic DNA from a transposon mutagenesis experiment was digested with the restriction endonuclease Tru91 (an isoschizomer of MseI) in a volume of 40 μl at 65° C. for 4 hours. The DNA was ethanol precipitated by adding 4 μl 3M NaOAc+200 μl 100% ethanol, mixed, centrifuged for 15 min (bench-top Eppendorf centrifuge), the supernatant removed and the remaining pellet washed with 200 μl 75% ethanol. The pellet was centrifuged for 5 minutes, the supernatant removed and the pellet vacuum dried for 10 minutes. The pellet was resuspended in 20 μl H₂O.

Following resuspension of the pellet, 1 μl of the DNA sample was run on a gel alongside 2 μl low mass markers to estimate quantity. The DNA sample was then diluted to a concentration of 200 ng in 100 μl of ligation mix [20 μl 5× ligation buffer, 5 μl ligase (5 units, GIBCO BRL) 75 μl DNA+H₂O]. The reaction was incubated for 2 hours at room temperature. The ligated DNA was ethanol precipitated as described above and resuspended in 10 μl H₂O.

Immediately following ligation, PCR was carried out with the PCR primer pair PHO2 and INV1 as follows:

1 μl of the above DNA in a 25 μl reaction mix:
12.5 μl Reddymix (PCR reaction mix, Abgene, UK)
9.5 μl H₂O
1 μl DNA
1 μl PHO2 primer (12 μM)
1 μl INV1 primer (12 μM)

PHO2 has the sequence:

5′-AGGTCACATGGAAGTCAGATCCTGG-3′ (SEQ ID NO: 4)

INV1 has the sequence:

5′-CTAAATCTGTGTTCTCTTCGGCGGC-3′ (SEQ ID NO: 5)

PCR was carried out under the following conditions: 95° C. for 5 min; 94° C. for 1 min; 64° C. for 1 min for 30 cycles, followed by 72° C. for 10 min. Following PCR, 5 μl of the PCR product was run on a gel for analysis.

One of the potential artifacts of the inverse PCR protocol is the inadvertent inclusion of a ‘stuffer’ fragment ligating into the self-ligation step outlined in step 3 above. Following PCR, the ‘stuffer’ fragment will be amplified along with the transposon-disrupted sequence. If this material were to be used in labeling experiments in the TMDH protocol, a non-specific background signal would be generated arising from the hybridization of the short ‘stuffer’ fragment to the polynucleotide library. In order to remove this ‘stuffer’ fragment the DNA can be redigested with Tru91 following PCR. If the transposon-gene junction important for the TMDH protocol is amplified by a biotin-labelled PHO2 primer, this fragment can conveniently be purified away from contaminating ‘stuffer’ fragments using a magnetic-bead-streptavidin conjugate. The purified DNA can then be labeled and used to hybridize to polynucleotide libraries, for example a gridded array.

(iii) Generating Specific Probes by Cycle Primer Extension

Cycle primer extension can be used to amplify fragments of DNA adjacent to the site of transposon insertion. The use of a labeled oligonucleotide primer in this procedure results in the generation of a specific hybridization probe.

50 μmol of the HPLC purified non-biotinylated PHO2 (right arm) primer (SEQ ID NO: 6) was labelled with 30 μCi [γ³³P] ATP using the forward reaction of the Gibco BRL 5 DNA labelling system as below with 10 units T4 polynucleaotide kinase in a 50 μl reaction volume (5 μl 10 pmole/μl HPLC purified PHO2 primer, 30 μl H₂O, 10 μl 5× forward reaction buffer, 3 μl 10 μCi/μl [γ³³P] ATP, 2 μl 5 units/μl T4 polynucleotide kinase).

Following incubation at 37° C. for 30 minutes the labeled primer was purified using the Qiagen Qiaquick Nucleotide Removal Kit. Labeled primer was recovered in a final volume of 30 μl.

To prepare the run-off template, E. coli genomic DNA containing a transposon in a known site (lamB) was purified using the Wizard Genomic DNA Purification Kit (Promega). The final concentration of the DNA was approximately 1 μg/ml. 20 μg of the genomic DNA was digested with 25 units of Tru91 at 65° C. for 2 hours and then digested for a further 2 hours after the addition of another 25 units of enzyme.

Following digestion, the DNA was electrophoresed and the gel fragment was excised that corresponded to between 200-500 bp. The DNA in this gel fragment was extracted using the Qiagen Gel Extraction Kit and eluted in a final volume of 301.

Run-offs were then generated using approximately 3 μg Tru91 digested 200-500 bp size selected DNA in a reaction mix consisting of 7 pmoles of labelled PHO2 primer, 0.2 mM dNTPs, and Boehringer Expand Taq polymerase (2 units) and buffer in a final volume of 100 μl.

The reaction conditions were an initial denaturation of 94° C. for 2 minutes followed by 60 cycles of 94° C. for 30 s, 55° C. for 30 s and 72° C. for 2 minutes.

Following the cycle primer extension reaction, the labeled product was hybridized to E. coli gridded array libraries.

1-28. (canceled)

29. A method for identifying an essential gene of an organism comprising:
(i) providing a library of transposon mutants of the said organism;
(ii) isolating from the library polynucleotide sequences flanking one side of the inserted transposons to give a first pool of sequences and polynucleotide sequences flanking the other side of the inserted transposons to give a separate second pool of sequences;
(iii) hybridising the first pool of sequences with a first sample of a polynucleotide library from the said organism and the second pool of sequences with a second sample of the said polynucleotide library from the said organism; and
(iv) identifying a polynucleotide in the said polynucleotide library to which at least one of the said pools of polynucleotide sequences does not hybridise, thereby to determine an essential gene of the organism.

30. A method according to claim 29, wherein step (iv) comprises identifying a polynucleotide in the said polynucleotide library to which the said pools of polynucleotide sequences do not hybridise, thereby to determine an essential gene of the organism.

31. A method according to claim 29, wherein the said polynucleotide library is in the form of a gridded array.

32. A method according to claim 29, wherein the organism is a bacterium, yeast, fungus, plant or animal.

33. A method according claim 29, wherein in step (ii) each pool of sequences is isolated by a method comprising:
(a) digesting genomic DNA isolated from a library of transposon-tagged mutants with a restriction endonuclease that cuts within the transposon (T-specific endonuclease) and a second different restriction endonuclease (G-specific endonuclease) which cuts within the disrupted sequence;
(b) ligating the resulting DNA fragments with a linker; and
(c) carrying out PCR on the resulting DNA fragments with an oligonucleotide specific for a transposon sequence and an oligonucleotide specific for a linker sequence.

34. A method according to claim 29, wherein the library of transposon mutants is a library of TnphoA E. coli mutants.

35. A method according to claim 33, wherein:
in the isolation of the first pool of sequences the restriction enzyme which cuts in the transposon is DraI and the second enzyme is a 4 base pair restriction endonuclease; and
in the isolation of the second pool of sequences the restriction enzyme which cuts in the transposon is HpaI and the second enzyme is a 4 base pair restriction endonuclease.

36. A method for identifying a conditional essential gene of an organism comprising:
(i) providing a first sample of a library of transposon mutants of the said organism (input library);
(ii) providing a second sample of the library and subjecting that sample to a conditional restraint;
(iii) collecting the mutants that survive the conditional restraint in step (ii) to give a new library (output library); and
(iv) carrying out a method according to claim 29 on the input library from step (i) and on the output library from step (iii), thereby to determine a conditional essential gene of the organism.

37. A method according to claim 36, wherein the organism is a bacterium and the conditional restraint is growth of that bacterium in its host.

38. A method for identifying an inhibitor of transcription and/or translation of an essential gene or a conditional essential gene of an organism and/or an inhibitor of activity of a polypeptide encoded by a said gene, which method comprises:
(a) identifying an essential gene or a conditional essential gene; and
(b) determining whether a test substance can inhibit transcription and/or translation of a gene identified in (a) and/or activity of a polypeptide encoded by a said identified gene, thereby to identify a said inhibitor.

39. An inhibitor identified by a method according to claim 38.

40. An inhibitor according to claim 39, wherein the essential or conditional essential gene is a bacterial, fungal or eukaryotic parasite essential or conditional essential gene.

41. A pharmaceutical composition comprising an inhibitor according to claim 40 and a pharmaceutically acceptable carrier or diluent.

42. A method for the preparation of a pharmaceutical composition, which method comprises:
(a) identifying an inhibitor of transcription and/or translation of an essential gene or conditional essential gene of an organism and/or an inhibitor of activity of a polypeptide encoded by a said gene, by a method according to claim 38, wherein the essential or conditional essential gene is a bacterial, fungal or eukaryotic parasite essential or conditional essential gene; and
(b) formulating an inhibitor identified in step (a) with a pharmaceutically acceptable carrier or diluent.

43. A method of treating a host suffering from a bacterial, fungal or eukaryotic parasite infection, which comprises administering to the host a therapeutically effective amount of an inhibitor according to claim 40.

44. An inhibitor according to claim 39, wherein the essential or conditional essential gene is a plant bacterial, plant fungal or plant pest essential or conditional essential gene.

45. A plant bactericide, plant fungicide or plant pesticide which comprises an inhibitor according to claim 44 and an agriculturally acceptable carrier or diluent.

46. An inhibitor according to claim 39, wherein the essential or conditional essential gene is a plant essential or conditional essential gene.

47. A herbicide which comprises an inhibitor according to claim 46 and an agriculturally acceptable carrier or diluent.

48. A bacterium attenuated by a non-reverting mutation in one or more genes identified by a method as defined in claim 37.

49. A method for the preparation of an attenuated bacterium, which method comprises:
(a) identifying a conditional essential gene in a bacterium by a method according to claim 37; and
(b) introducing a non-reverting mutation into a conditional essential gene identified in (a) of the bacterium, thereby to attenuate the bacterium.

50. A vaccine comprising a bacterium according to claim 48 and a pharmaceutically acceptable carrier or diluent.

51. A method for the preparation of a vaccine, which method comprises:
(a) identifying a conditional essential gene in a bacterium by a method according to claim 37;
(b) introducing a non-reverting mutation into a conditional essential gene identified in (a) of the bacterium, thereby to attenuate the bacterium; and
(c) formulating the attenuated bacterium prepared in (b) with a pharmaceutically acceptable carrier or diluent.

52. A method of raising an immune response in a mammalian host, which comprises administering to the host a bacterium according to claim 48.

53. A method of raising an immune response in a mammalian host, which comprises administering to the host a vaccine according to claim 50.

54. A method for raising an immune response in a host, which method comprises:
(a) identifying a conditional essential gene in a bacterium by a method according to claim 37;
(b) introducing a non-reverting mutation into a conditional essential gene identified in (a) of the bacterium, thereby to attenuate the bacterium;
(c) formulating the attenuated bacterium prepared in (b) with a pharmaceutically acceptable carrier or diluent; and
(d) administering to the host the attenuated bacterium formulated in (c).


SEQUENCE LISTING (5 sequences)

SEQ ID NO: 1 - DNA, 20 bp, Artificial Sequence
  Description of Artificial Sequence T7 RNA polymerase site
taatacgactcactataggg

SEQ ID NO: 2 - DNA, 25 bp, Artificial Sequence
  Description of Artificial Sequence PCR primer
ccggctcgtgtcgactgtggaattg

SEQ ID NO: 3 - DNA, 27 bp, Artificial Sequence
  Description of Artificial Sequence PCR primer
ctgcaggcatgcaagctttccctatag

SEQ ID NO: 4 - DNA, 25 bp, Artificial Sequence
  Description of Artificial Sequence PCR primer
aggtcacatggaagtcagatcctgg

SEQ ID NO: 5 - DNA, 25 bp, Artificial Sequence
  Description of Artificial Sequence PCR primer
ctaaatctgtgttctcttcggcggc